(19) United States
(12) Patent Application Publication
(10) Pub. No.: US 2023/0149324 A1
(43) Pub. Date: May 18, 2023

(54) COMPOSITIONS COMPRISING THYMOL AND AMINO ACIDS FOR USE IN THE TREATMENT OF INFLAMMATORY OR FUNCTIONAL INTESTINAL DISORDERS

(71) Applicant: VETAGRO INTERNATIONAL S.R.L., Reggio nell'Emilia (IT)

(72) Inventors: Andrea PIVA, Reggio nell'Emilia (IT); Ester GRILLI, Reggio nell'Emilia (IT)

(21) Appl. No.: 18/014,738

(22) PCT Filed: Jul. 26, 2021
(86) PCT No.: PCT/IB2021/056723

§ 371 Date: Jan. 5, 2023

(30) Foreign Application Priority Data
Jul. 31, 2020 (IT) 102020000018913

(51) Int. Cl. A61K 31/05; A61K 45/06; A61K 31/405; A23K 10/30; A61P 29/00

(52) U.S. Cl. CPC A61K 31/05; A61K 45/06; A61K 31/405; A23K 10/30; A61P 29/00

(57) ABSTRACT

The present invention relates to compositions comprising thymol and at least one amino acid for use in the preventive and/or curative treatment of inflammatory or functional diseases or symptoms of the intestinal tract by modulating the receptors and/or enzymes of the endocannabinoid system in mammalian or non-mammalian monogastric subjects, such as human subjects, pigs, poultry animals, fish or crustaceans.

The present invention relates to compositions comprising thymol for use in the preventive and/or curative treatment of inflammatory or functional diseases of the intestinal tract and related symptoms in human subjects, or monogastric animals (mammalian and/or non-mammalian), or poultry or fish, by modulating the receptors and/or enzymes of the endocannabinoid system.

Furthermore, the present invention relates to compositions comprising thymol and at least one amino acid for use in the preventive and/or curative treatment of inflammatory or functional diseases or symptoms of the intestinal tract in human subjects, or monogastric mammals (for example pigs) or non-mammalian monogastric animals (such as, poultry animals, fish or crustaceans), by modulating the receptors and/or enzymes of the endocannabinoid system.

Inflammatory or functional diseases or symptoms of the intestinal tract are widely common both in humans and in monogastric animals (mammalian or non-mammalian), negatively affecting their development—in particular in human or animal subjects in the growth phase—their quality of life and exposing them to the risk of developing further diseases due to the weakening of the immune system.

Among these, of particular importance are chronic inflammatory bowel diseases (IBDs), a group of nosological entities characterised by the presence of chronic phlogosis in the absence of infectious aetiology (i.e. not caused by bacteria, viruses or fungi). The two most important diseases of the group are Crohn's disease Crohn and ulcerative rectocolitis. IBD is a multifactorial disease, driven in part by an exaggerated immune response at the gut microbiota level that causes defects in the epithelial barrier function. In particular, the loss of integrity of the intestinal epithelium plays a key pathogenic role in IBD. Most animals with IBD have a history of recurrent or chronic vomiting and/or diarrhoea. Often, a significant weight loss may be observed in animals affected by IBD, with all the entailed consequences.

Another gastrointestinal disease strongly common in humans and even in the monogastric animal population is irritable bowel syndrome (in short, IBS). Irritable bowel syndrome belongs to the group of functional gastrointestinal disorders (FGIDs), a diagnostic category that can be defined on the basis of the symptom presentation alone and characterised by the absence of an evident pathogenetic substrate (i.e. absence of bacteria, viruses or mycetes), in which abdominal discomfort or pain is associated with modifications of the gut microbiota. Depending on the characteristics of the faeces, four groups of the disease are distinguished: IBS with predominant constipation (constipated bowel), IBS with predominant diarrhoea (diarrhoeic bowel), IBS with alternating constipation and diarrhoea, unclassified IBS.

In addition, in animals with damaged or inflamed intestinal epithelium, the intestinal action for the absorption of nutrients (for example, amino acids) is impaired, given that the intestinal epithelium is the area in which enterocytes responsible for the absorption of nutrients reside.

Inflammation of the intestinal epithelium can be caused by stress (for example, as regards animals stress caused by mass farming) and by inflammatory insults of various kinds (such as, harmful ingredients present in the diet, pathogenic infections, environmental stress such as heat-related stress, etc.).

The drugs or compounds available to date for the treatment of inflammatory or functional diseases or symptoms of the intestinal tract in humans or monogastric animals often do not allow a complete and/or lasting resolution of the disease and the symptoms thereof. Furthermore, the drugs and treatments available on the market are not free of unwanted side effects which therefore sometimes limit their use as well.

Therefore, the interest on the part of the operators in the industry in being able to have a treatment that allows to overcome the limits and drawbacks still present in the known treatments and that is able to provide a rapid, effective and durable response against inflammatory or functional diseases or symptoms of the intestinal tract in humans and in monogastric animals (such as pigs, birds, chickens, fish and crustaceans)

The technical problem addressed and solved by the present invention lies in providing a composition and effective treatment for the treatment of intestinal disorders (diseases or symptoms) both of inflammatory and functional nature, mainly in the absence of bacterial, viral or fungal aetiology, in human subjects, in monogastric animals, preferably monogastric mammals, such as for example pigs or domestic animals, in poultry (non-mammalian monogastric animals), preferably birds and chickens, and in fish or crustaceans (non-mammalian monogastric animals), particularly also regarding the maintenance of a health condition and well-being of the intestine and/or in the support or increase in the weaning (or initial growth) phase.

Furthermore, the technical problem addressed and solved by the present invention lies in treating inflammatory and/or functional intestinal diseases or symptoms, in order to guarantee the subject an intestinal epithelium that is intact and efficient in the digestion and absorption of nutrients, such as for example amino acids.

An intestinal epithelium efficient in the absorption of amino acids administered to a monogastric subject allows to treat a deficiency of amino acids in said subjects. In order to effectively treat a deficiency of amino acids, the administration of amino acids alone is not sufficient, given that in the presence of an inflamed intestine, the organism of the subject is not able to absorb (fully or partly) said administered amino acids.

Providing human subjects or monogastric animals with a balanced supply of amino acids (which varies depending on the subject, the age of the subject and other factors) means supporting the growth thereof and treating, in a preventive or curative manner, conditions of reduced muscle mass and/or strength and malnutrition conditions (such as for example celiac disease or sarcopenia). Providing human subjects or monogastric animals with a balanced supply of amino acids means reducing the presence of excess proteins in the intestine of said subjects and, therefore, the possibility of bacterial infections. Providing monogastric animals with a balanced supply of amino acids means reducing the nitrogen excretion of said animals and, therefore, the environmental impact of the animal farms. Providing human subjects or monogastric animals with a balanced supply of amino acids means reducing the percentage of protein in the diet of the subject, leading to an economic advantage, in particular in the case of animals and their mass farming.

In addition, the technical problem addressed and solved by the present invention lies in providing said amino acids so that the blood bioavailability thereof is constant over a period of time ranging from 2 hours to 24 hours, so as to avoid fluctuations of said bioavailability between one meal and the other.

Following an intense research and development activity, the Applicant addresses and solves the aforementioned technical problem by providing innovative mixtures, compositions (in short, mixtures and compositions of the invention) and treatments, having the characteristics as defined in the description and in the attached claims.

The present invention is also better disclosed with the aid of the following figures, provided solely by way of example and therefore not limiting the scope of protection thereof.

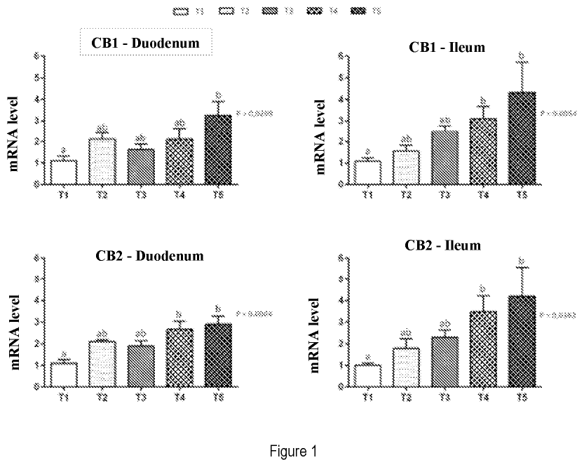

FIG. 1 reports the gene expression data for cannabinoid receptors in the duodenal and ileal mucosa at day 14 (d14) in pigs.

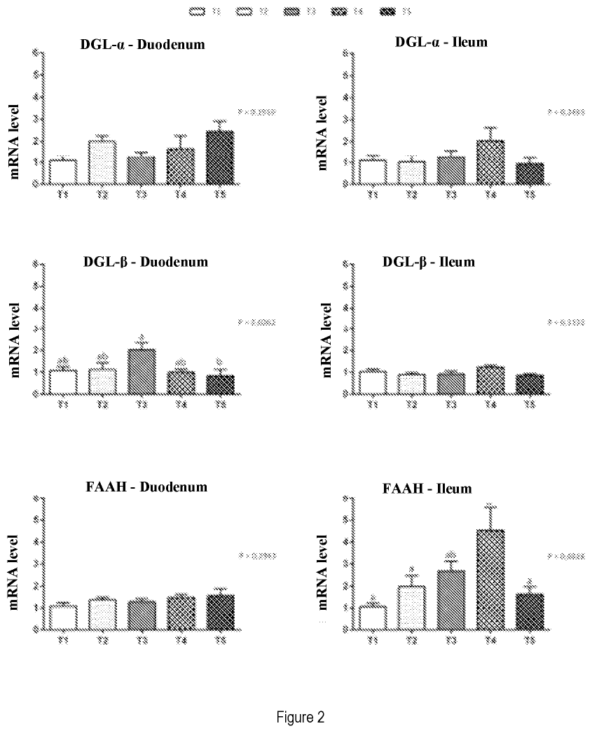

FIG. 2 reports the gene expression data for ECS enzymes in the duodenal and ileal mucosa at day 14 (d14) in pigs.

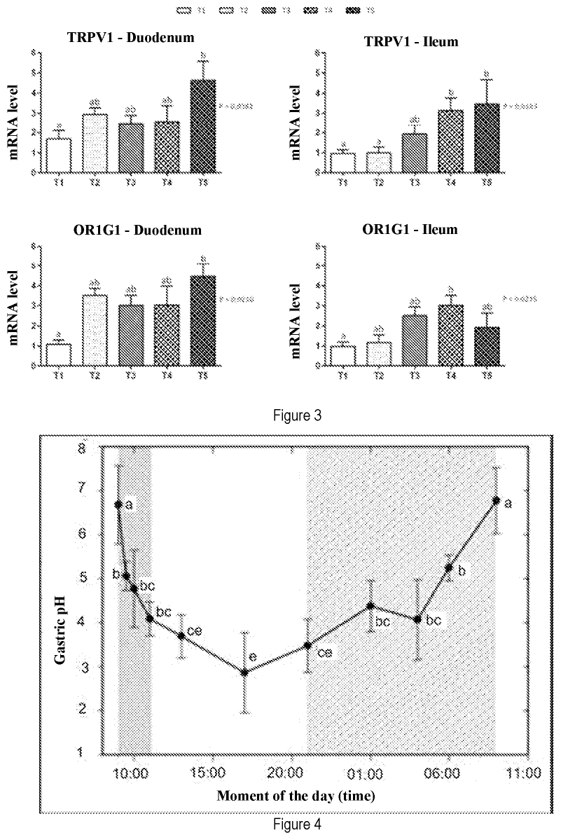

FIG. 3 reports the gene expression data for gut chemosensing in the duodenal and ileal mucosa at day 14 (d14) in pigs.

FIG. 4 reports the gastric pH (mean±DS) during the 24-hour cycle in the young ones of Diplodus sargus (family of Teleostei). The same letter does not indicate any significant difference (P>0.05). Dark grey area (about 10.00 hrs): feeding time. Light grey area (about 21.00-8.00 hrs): dark period.

### DETAILED DESCRIPTION OF THE INVENTION

A first aspect of the present invention relates to a composition according to a first embodiment (in short, FR-I), wherein said composition comprising a (i) mixture M of active components and (iii) at least one acceptable pharmaceutical or food grade additive and/or excipient, and wherein said (i) mixture of active components comprises or, alternatively, consists of thymol. Preferably, said composition according to FR-I further comprises (ii) a controlled release lipid matrix.

A second aspect of the present invention relates to a composition according to a second embodiment (in short, FR-II), wherein said composition comprising a (i) mixture M of active components and (iii) at least one acceptable pharmaceutical or food grade additive and/or excipient, and wherein said (i) mixture of active components comprises or, alternatively, consists of thymol and at least one amino acid. Preferably, said composition according to FR-I further comprises (ii) a controlled release lipid matrix.

A third aspect of the present invention relates to a composition (composition according to FR-I or FR-II) for use (in short, composition of the invention) in a method for the preventive and/or curative treatment of an inflammatory and/or functional intestinal disease or of a related symptom by modulating (or increasing gene expression) the receptors and/or enzymes of the endocannabinoid system and/or of the gut chemosensing system in a human being, or in a monogastric animal (mammalian or non-mammalian, for example in a pig, or in a bird, or in a chicken, or in a fish, or in a crustacean), wherein said composition comprises a mixture M (in short, mixture M of the invention) comprising or, alternatively, consisting of thymol (according to FR-1) or thymol and at least one amino acid (according to FR-II), and optionally, the composition further comprises at least one acceptable pharmaceutical or food grade additive and/or excipient.

The inflammatory and/or functional intestinal disease, or related symptom, is selected from intestinal diseases which do not mainly have microbial aetiology (bacterial, viral or fungal). Advantageously, the compositions of the present invention are validly used in the treatment of said diseases.

Advantageously, the compositions subject of the present invention (FR-1 and FR-II) show interesting therapeutic properties for the preventive and/or curative treatment of inflammatory or functional diseases of the intestinal tract thanks to the ability of thymol to modulate the intestinal gene expression of receptors of the endocannabinoid system (ECS), such as cannabinoid-1 (CB1) and cannabinoid-2 (CB2), and/or the enzymes of the endocannabinoid system (ECS), such as for example the fatty acid amide hydrolase (FAAH) involved in the degradation of the endocannabinoid molecule anandamide (AEA), and, in addition, to modulate one or more markers of the gut chemosensing system, such as for example transient receptor potential vanilloid-1 (TRPV1) and/or olfactory receptor 1G1 (OR1G1).

Advantageously, the compositions developed by the Applicant can be formulated and prepared so as to allow a gradual and targeted release into the intestine as a function of the species treated. A gradual release of thymol and of further phytocompound derivatives, if present, in the various portions of the gastrointestinal tract over time enhances the efficacy of the antioxidant, anti-inflammatory and immunostimulant activity thereof and, therefore, the efficacy thereof in maintaining or restoring the homeostasis and the health of the microbiota of the intestinal mucosa. In addition, thymol and said further phytocompound derivatives described in the present invention show an antibacterial activity which contributes to intestinal health. Table A reports—regarding chickens—an example of the transit time of the feed upon variation of the pH in the various segments of the digestive tract. As regards prawns and crustaceans, the transit time of the feed in the intestinal tract is instead comprised from 30 minutes to 120 minutes, preferably from 40 minutes to 90 minutes.

TABLE A

| Segment of the digestive tract of broilers | pH | Transit time (min) |
|---|---|---|
| Gizzard | 5.5 | from 10 to 50 |
| Proventriculus/Ventriculus | 2.5-3.5 | from 30 to 90 |
| Duodenum | 5-6 | from 5 to 10 |
| Fasting | 6.5-7.0 | from 20 to 30 |
| Ileum | 7.0-7.5 | from 50 to 70 |
| Cecum/colon | 8.0 | from 20 to 30 |

According to a preferred aspect of said composition of the present invention, besides thymol (according to FR-I) or thymol and at least one amino acid (according to FR-II), the composition comprising a controlled release lipid matrix embedding thymol and possible other active components present in the composition of the invention (for example, other phytocompound derivatives, organic acids and/or amino acids). Said controlled-release lipid matrix allows a gastroprotection (a protection against the acidic pH of the stomach) and a controlled release of thymol and other active components (for example, other phytocompound derivatives, organic acids and/or amino acids), possibly present in the composition of the invention, in the intestine over time (from 30 minutes to 8 hours, preferably 1 hour to 6 hours).

The lipid matrix of the invention is stable at the predominantly acidic pH of the stomach (or gizzards in the case of birds): pH 2-6, depending on the digestive phase (FIG. 4, pH of fish). As a result, the lipid matrix by embedding and/or incorporating thymol and, if present, the other active components (for example, amino acids and further phytocompound derivatives), allows the transit thereof through the stomach without undergoing degradation. When the composition of the invention reaches the intestine and/or the hepatopancreas, where the pH has a higher value with respect to the stomach (pH 4-7) and where there are present enzymes capable of digesting lipid substances (i.e. lipases), the lipid matrix dissolves slowly allowing a controlled release of the active components (such as amino acids and phytocompound derivatives).

Furthermore, when the composition of the invention comprises at least one amino acid (according to FR-II), the presence of said lipid matrix provides, following an oral administration of the composition of the invention, a blood (or plasma) bioavailability of said at least one amino acid in a constant percentage over a period of time comprised from 2 hours to 24 hours.

Activation of the endocannabinoid system (ECS) (receptors, signalling molecules and enzymes involved in ligand biosynthesis and degradation) is believed to be a valid approach in the control and in the regulation of inflammation and the functioning of the gastrointestinal system.

Cannabinoid receptors, whose main representatives are CB1 and CB2, are present in most of the organism including also in cells located in the intestine and digestive system. It has therefore been suggested that ECS regulates important physiological processes, including immune response, metabolism, digestive motility and appetite, and that it contributes to the maintenance of homeostasis, the sensitive internal balance of the organism. As a result, an irregular functioning of the endocannabinoid system, in particular modulation of the gene expression of receptors (CB1 and CB2) and enzymes, plays a decisive role in inflammatory and functional intestinal diseases.

Furthermore, it has been suggested that the “gut chemosensing system” may have therapeutic applications in the treatment of intestinal inflammatory or functional diseases due to the intestinal presence of a large variety of receptors, such as for example TRPV1 (transient receptor potential vanilloid 1) and OR1G1 (olfactory receptor 1G1).

The compositions or mixtures of the present invention have no significant side effects and they can be administered both to adult human subjects and animals, to human subjects and animals in the growth phase and to pregnant female animals.

Lastly, the compositions or mixtures of the present invention are easy to prepare and cost-effective.

When the composition of the invention does not include additives and/or excipients, the composition of the invention is identical to the mixture M of the invention.

Said treatment method of the present invention provides for the administration of a therapeutically effective amount of the composition or mixture M of the invention to the human subject, monogastric animal, poultry or fish.

In the context of the present invention, the expression “monogastric animals” is intended to include both mammalian and non-mammalian monogastric animals.

Furthermore, the composition or mixture M of the present invention comprising thymol (and preferably other active compounds, such as, for example, phytocompound derivatives, organic acids and/or amino acids, according to FR-I or FR-II) is for use in maintaining or increasing the health of the intestine and/or for supporting in the weaning (or initial growth) phase of said human subjects or monogastric animals (mammalian and non-mammalian), preferably monogastric mammals or poultry or fish.

The expression “support” or “support in the weaning or initial growth phase”, in the context of the present invention is used to indicate the supply of compounds (such as phytocompound derivatives, organic acids and/or amino acids) in order to treat a deficiency of said compounds or in order to treat a disease, symptom or disorder deriving from such deficiency in a subject.

Preferably, said monogastric animal is a monogastric mammal, in particular a monogastric animal or monogastric mammal in the weaning phase. Furthermore, in the context of the present invention non-mammalian monogastric animals, such as poultry animals, such as for example birds and chickens, fish and crustaceans are also included.

Said monogastric mammals, adult or in the weaning phase, which can be treated by means of the compositions or mixtures M of the invention, may be selected from: dogs, cats, monkeys, pigs, equines, such as horses and donkeys, rabbits before weaning, rodents, such as hamsters, cavies, mice, gerbils, chinchillas, degus, squirrels, guinea pigs and rats; weasels, ferrets and ermines, preferably pigs.

Said non-mammalian monogastric animals, adult or in initial growth phase, that can be treated using the compositions or mixtures M of the invention, can be selected from: animals of the poultry species (class Aves, preferably order Galliformes), such as chicken or other poultry, turkey, guinea fowl, pheasant, peacock, partridge, quail, dove, turtle dove, goose, common duck and Muscovy duck (preferably, chicken, laying hen, turkey, or, alternatively, an animal belonging to the aquatic species, such as fish (a fish which can be bred in fresh water or which can be bred in salty water, for example salmon, trout, seabass, gilthead bream, tilapia, carp, catfish and the like) and crustaceans (farm crustaceans for example prawns); preferably chickens, laying hens, turkey and fish.

The diseases or symptoms that can be preventively and/or curatively treated using the compositions or mixtures M of the invention, according to any one of the described embodiments, are advantageously selected from: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, celiac disease, irritable bowel syndrome (IBS), IBS with diarrhoea, IBS with constipation, IBS with alternating constipation and diarrhoea, unclassified IBS, dyspepsia, nausea, vomiting, constipation, diarrhoea, abdominal bloating, tympanites and physical fatigue.

The compositions or mixtures M of the present invention, according to any of the described embodiments, may be for use as adjuvants of further therapeutic approaches (e.g. drugs) in the treatment of the diseases indicated in the present invention.

Besides thymol (according to FR-I) or besides thymol and at least one amino acid (according to FR-II), the mixture M, contained in the composition of the invention (in the presence or in the absence of the (ii) lipid matrix), may further comprise a further first active ingredient deriving from a phytocompound (botanicals), selected from the group (I) comprising or, alternatively, consisting of carvacrol, eugenol, capsaicin, turmeric, vanillin, cinnamaldehyde, diallyl disulfide, camphor, limonene, rosmarinic acid, p-cymene, γ-terpinene, α-pinene, α-thujone, 1,8-cineole, verbascoside, tannins, saponins and mixtures thereof. Preferably said further first active ingredient deriving from a phytocompound (botanicals) is selected from the group (I.i) comprising or, alternatively, consisting of: (b) carvacrol, (c) eugenol, (d) capsaicin, (e) tannins, (f) verbascoside, (g) saponins and mixtures thereof.

For example, according to FR-I, the composition of the invention may comprise thymol and carvacrol, or thymol and vanillin, or thymol, carvacrol and capsaicin, or thymol, carvacrol and cinnamaldehyde, or thymol, eugenol and verbascoside.

Besides thymol (according to FR-I) or besides thymol and at least one amino acid selected from group (IV) (according to FR-II) and, optionally, said further first active ingredient (botanicals) selected from group (I), the mixture M, contained in the composition of the invention may further comprise an organic acid or a salt thereof with an alkaline or alkaline earth metal cation, wherein said organic acid is selected from group (II) comprising or, alternatively, consisting of lactic acid, malic acid, benzoic acid, fumaric acid, sorbic acid, citric acid, octanoic acid, heptanoic acid, butyric acid, dodecanoic acid and mixtures thereof. For example, the composition of the invention may comprise thymol and sorbic acid; thymol, sorbic acid and citric acid; thymol and benzoic acid; thymol, sorbic acid, citric acid and benzoic acid; thymol, carvacrol and sorbic acid; thymol, carvacrol, sorbic acid and citric acid; thymol, carvacrol, cinnamaldehyde and sorbic acid, or thymol, carvacrol, cinnamaldehyde, sorbic acid and citric acid, thymol and butyric acid, thymol, citric acid and dodecanoic acid.

In the mixture M, contained in the composition of the present invention, the molar ratio between A and B, wherein: A is thymol and, optionally, at least one or more of said further first active ingredient (botanicals) selected from group (I), while B is at least one or more organic acids or salts thereof selected from group (II), is comprised in the range from 1:500 to 500:1, preferably from 1:300 to 300:1, more preferably from 1:100 to 100:1 or from 1:50 to 50:1 or from 1:10 to 10:1.

Besides thymol (according to FR-I) or besides thymol and at least one amino acid selected from group (IV) (according to FR-II) and, optionally, said further first active ingredient selected from group (I) and/or said organic acid or a salt thereof selected from group (II), according to any one of the described embodiments, the mixture M contained in the composition of the invention (in the presence or in the absence of the (ii) lipid matrix) may further comprise at least one further second active ingredient selected from group (III) consisting of:
bacterial strains or probiotic bacterial strains belonging to the genus Lactobacillus, Streptococcus, Leuconostoc, Bifdobacterium, Pediococcus, Enterococcus, Saccharomyces;
prebiotics, such as for example inulin, lactulose, lactitol, mannan-oligosaccharides, fructigosaccharides and galacto-oligosaccharides, tributyrin;
salts of metals, such as zinc and copper; and mixtures thereof.

Furthermore, the composition of the present invention may comprise a (ii) controlled release lipid matrix embedding or incorporating said mixture M which comprises (according to FR-I) or thymol and at least one amino acid selected from group (IV) (according to FR-II) and, optionally, further ingredients selected from group (I) and/or (II) and/or (III), according to any one of the described embodiments. Said controlled release lipid matrix comprises or, alternatively, consists of at least one saturated or unsaturated, free or esterified fatty acid, having a number of carbon atoms comprised in the range from C10-C30, preferably from C14-C24 or C16-C22, and/or at least one triglyceride having saturated or unsaturated fatty acid chains having a number of carbon atoms comprised in the range from C6-C30, preferably from C14-C24 or C17-C21, and/or at least one wax having a number of carbon atoms comprised in the range from C16-C36, preferably from C24-C36 or C26-C32; wherein said lipid matrix allows a gastroprotection from the acidic pH of the stomach and ensures an intestinal controlled release of thymol and of the further components possibly present in the mixture M, with release times as a function of the species to be treated and of the type of lipid matrix used.

The mixture M and the composition containing it are prepared using techniques and equipment known to the person skilled in the art. In particular, the process for preparing the mixture M and the composition thereof comprising the lipid matrix provides for mixing, in a mixer or in a container provided with stirring (or mixing) and heating means, thymol and, optionally, one or more of the further ingredients selected from group (I) and/or (II) and/or (III) (according to any one of the described embodiments), together with the lipid matrix so that all the compounds and ingredients are embedded together in the matrix as a whole. Preferably, the mixture M or the composition of the invention (both according to FR-I and according to FR-II), is obtained through the preparation process described in the patent application n° EP 1 391 155 A1 paragraphs [0048]-[0049] and [0077]; said paragraphs are incorporated in the present description for reference.

“Triglycerides” (or triacylglycerols) are neutral esters of glycerol in which chains of three long chain fatty acids are present instead of the hydrogen atoms of the hydroxyl groups. The length of the fatty acid chains in the common triglycerides structures may be from 5 to 28 carbon atoms, but 17 and 19 are more common.

The term “fatty acids” (in short FAs) is mainly but not exclusively used to indicate long-chain aliphatic monocarboxylic acids (number of carbon atoms comprised in the range C10-C30) with an even number of carbon atoms, without branching and acyclic (i.e., consisting of molecules that do not have ring-like closed chains). Fatty acids can be saturated (if their molecule has single C—C bonds only) or unsaturated (if they have double C═C bonds).

The term “waxes” is used to indicate to long-chain fatty acid esters with high molecular weight monohydric alcohols. Waxes may be of plant origin or animal origin (beeswax). Beeswax consists of various compounds, including: hydrocarbons 14%, monoesters 35%, diesters 14%, triesters 3%, hydroxy monoesters 4%, hydroxy polyesters 8%, acid esters 1%, acidic polyesters 2%, free acids 12%, free alcohols 1%, not identified 6%. The main components of beeswax are palmitates, palmitic acid, hydroxypalmitates and oleate esters formed by long chains (30-32 carbon atoms) of aliphatic alcohols, with a 6:1 ratio between the two main components triacontanyl palmitate (myricyl palmitate) CH₃(CH₂)₂₉O—CO—(CH₂)14 CH₃ and cerotic acid CH₃(CH₂)₂₄COOH. Beeswax has a melting comprised between 62° C. and 64° C. Density at 15° C. ranges between 0.958 g/cm³ and 0.970 g/cm³. Beeswax can be classified into two broad categories: European type and Eastern type. The saponification number is 3-5 for European type and 8-9 for Eastern type.

Said triglyceride comprised in said controlled release lipid matrix may be a hydrogenated or non-hydrogenated triglyceride of plant or animal origin, preferably hydrogenated triglyceride of plant and/or animal origin, more preferably a hydrogenated triglyceride of plant origin.

Said fatty acid comprised in the controlled release lipid matrix may be a hydrogenated or non-hydrogenated fatty acid, or an ester thereof of plant and/or animal origin, preferably a hydrogenated fatty acid of plant and/or animal origin, more preferably a hydrogenated fatty acid of plant origin.

Said waxes comprised in the controlled release lipid matrix may be of plant and/or animal origin; preferably beeswax.

Examples of said fatty acid, triglyceride or wax of plant origin, even in the hydrogenated form, are: palm oil, sunflower oil, corn oil, rapeseed oil, peanut oil, soybean oil, olive oil, beeswax, and mixtures thereof; preferably rapeseed oil or palm oil or soybean oil or a mixture thereof.

Examples of triglycerides of animal origin, also in the hydrogenated form, are selected from: chicken fat, hydrogenated chicken fat, bovine tallow and pork lard.

According to a preferred aspect of said second embodiment (FR-II) of the present invention, the composition of the invention comprises: (i) said mixture M of the invention comprising thymol and at least one amino acid selected from group (IV) comprising or, alternatively, consisting of: lysine (Lys), methionine (Met), tryptophan (Trp), threonine (Tre), valine (Val), leucine (Leu), isoleucine (Iso-Leu), arginine (Arg), histidine (His), phenylalanine (Phe); the composition of the invention further comprises (ii) said controlled release lipid matrix comprising or, alternatively, consisting of: (ii.1) a triglyceride, (ii.2) an organic acid, (ii.3) a wax or a mixture thereof (as defined in the context of the present invention.

For example, the composition of the invention comprises (i), (ii) and, optionally, (iii), wherein: said (i) mixture M of the invention comprises thymol and at least one amino acid selected from group (IV), and optionally, phytocompound derivatives of group (I.i) and/or organic acids of group (II); and (ii) said lipid matrix comprises or, alternatively, consists of a vegetable oil selected from the group consisting of: rapeseed oil, palm oil, soybean oil or a mixture thereof; preferably rapeseed oil when said monogastric animal is poultry, fish or crustacean; preferably soybean oil when said monogastric animal is a pig or a human subject.

The composition of the invention, may further comprise the mixture M, comprising thymol and, optionally, at least one further ingredient selected from group (I) or (I.i), (II) and/or (III) and/or (IV) according to any one of the described embodiments (for example thymol and at least one amino acid selected from group IV), in a % by weight comprised in the range from 1% to 80% (for example, 5%, 20%, 25%, 30%, 40%), preferably from 5% or 10% to 50%, more preferably from 15% to 45%, and said controlled release lipid matrix, according to any one of the described embodiments, in a % by weight comprised in the range from 10% to 80% (for example, 20%, 30%, 40% or 50%); preferably from 40% to 60% or from 30% to 70%, more preferably from 45% to 55%, wherein said % are with respect to the total weight of the composition.

According to an aspect of said second embodiment of the invention (FR-II, for example from FR-II-1 to FR-II-22), the composition of the invention comprises: said at least one amino acid from 1% to 80% (for example 5%, 10%, 15%, 20%, 25% or 30%), preferably from 5% to 40%, more preferably from 5% to 35%; thymol and, optionally, a further phytocompound derivative selected from group (I) from 5% to 15% (for example 5%, 10%, 15%, 20%, 25% or 30%), preferably from 1% to 10%, more preferably from 1% to 5%, said (ii) lipid matrix from 10% to 80% (for example 15%, 20%, 25%, 35%, 50% or 65%); preferably from 30% to 70%, more preferably from 45% to 55%, and, optionally, said (iii) additive and/or excipient from 0.1% to 30% (for example 0.5%, 2%, 4%, 6%, 8%, 15% or 25%), preferably from 1% to 20%, more preferably from 5% to 10%; wherein said percentages are percentages by weight with respect to the total weight of the composition.

The composition of the invention, according to any of the described embodiments, may be a feed or a feed additive.

Forming an object of the present invention is a feed comprising the composition of the present invention (according to any one of the described embodiments or aspects) and nutrients suitable, according to the person skilled in the art, for the type of monogastric animal (mammalian or non-mammalian) for which said feed is intended, preferably pigs, chickens, laying hens, turkey, fish and crustaceans.

Alternatively, said composition of the invention, according to any one of the described embodiments, may be a pharmaceutical composition, a medical device composition, (EU Regulation 2017/745 (MDR) a dietary supplement, a food (or novel food) (EC Regulation 258 of 1997) or a food for special medical purposes, a composition for a dietary or food supplement, both for human subjects and for animals (veterinary products).

Advantageously, the mixtures or compositions of the present invention, according to any of the described embodiments, are formulated for oral use.

The composition of the present invention may be formulated for oral use in solid form, for example, granules, flakes, powder, soluble powder or granules, tablets, capsules; or, alternatively, in liquid form, for example, selected from: solutions, suspensions, emulsions, liquid which can be dispensed in the form of sprays, syrups; or, alternatively, in semi-liquid form, for example, selected from: soft-gels, gels.

Preferably the composition of the invention is for oral use in solid form, for example granules, powder or flakes.

According to an aspect both of said first embodiment (FR-I) and said second embodiment (FR-II) of the invention, the composition of the invention comprising (i), (ii) and, optionally, (iii), is in form of relatively spherical particles (such as granules or microcapsules) having an average particle size (or particle size) comprised in the range from 50 μm to 2500 μm for example 250 μm, 400 μm, 500 μm, 1500 μm, to 2000 μm.

Within a batch of composition of the invention (according to FR-I or FR-II preferably from FR-1 to FR-22,) in the form of granules, the granules have different particle size with a particle size distribution percentage within the above indicated particle size ranges. Said particle size distribution percentage may vary depending on whether the composition is for use in the treatment of a deficiency of amino acids in human subjects, pigs, poultry animals, fish or crustaceans.

For pigs or human subjects, a batch of 100 granules of the composition may have the following particle size distribution percentage: from 25% to 35% of granules has a particle size from 500 μm to 1000 μm, from 45% to 55% 1000 μm-1500 μm, from 20% to 30% 1500 μm-2000 μm, from 0.1% to 1% 2000 μm-2500 μm (wherein said percentages are percentages of granules with respect to 100 granules).

For animals of the aquatic species (fish and crustaceans), a batch of 100 granules of the composition may have the following particle size distribution percentage: from 10% to 20% of granules has a particle size from 50 μm to 250 μm, from 45% to 55% 250 μm-400 μm, from 20% to 30% 400 μm-500 μm, from 5% to 15% 500 μm-2500 μm (wherein said percentages are percentages of granules with respect to 100 granules).

For subjects of the poultry species (e.g. chickens, laying hens, turkeys), a batch of 100 granules of the composition may have the following particle size distribution percentage: from 1% to 10% of granules has a particle size from 50 μm to 500 μm, from 45% to 55% the particle size measures 500 μm-1000 μm, from 35% to 45% the particle size measures 1000 μm-1500 μm, from 1% to 9% the particle size measures 1500 μm-2000 μm, from 0.1% to 1% the particle size measures 2000 μm-2500 μm (wherein said percentages are percentages of granules with respect to 100 granules).

Examples of batches of the composition of the invention (according to FR-I or FR-II) in the form of granules are reported in Table B. Said particle size percentage distribution is constant and reproducible in the preparation of the batches of the composition of the invention. Upon reaching the intestine, said granules break up at different times and in different sections of the intestine depending on their granulometry. Thus, the effect of said particle size distribution percentage consists in a slow and progressive release of the active compounds comprised in the mixture M embedded in the lipid matrix along the whole section of the intestine. In particular, the smaller granules are digested (releasing the active ingredients) over a short period of time in the upper part of the intestine, whereas larger granules are digested by lipases more slowly and the release of active ingredients occurs over a longer period of time with respect to the smaller granules and more distal position along the intestinal tract.

TABLE B
P-C: fish and crustaceans. U-M: man and pig

| | | | | | | |
|---|---|---|---|---|---|---|
| 50-250 | 250 ÷ 400 | 400 ÷ 500 | 500 ÷ 1000 | 1000 ÷ 1500 | 1500 ÷ 2000 | 2000-2500 |
| um | um | um | um | um | um | um |

| | | | | |
|---|---|---|---|---|
| P-C | 15% | 50% | 25% | 10% |

| | | | | | |
|---|---|---|---|---|---|
| poultry | 5% | 50% | 40% | 4.5% | 0.5% |
| U-M | 5% | 30% | 40% | 24.5% | 0.5% |

The compositions of the invention comprising said lipid matrix and formulated in solid form of granules have an optimal gradual release of the active components comprised in the composition (thymol, phytocompound derivatives and/or amino acids) in the various sections of the intestine over time. Said advantage partly arises from the particle size of the composition of the invention (or particle size percentage distribution), and from the characteristic of the granules of the composition of the invention to disintegrate at different times and in different sections of the intestine as a function of the particle size thereof.

To define the particle size percentage distribution of a batch of the composition of the present invention, instruments and methodologies known to the man skilled in the art can be used for particle size analysis. For example, within the scope of the present invention, one of the following two methods can be used to define said particle size distribution percentage: particle size analysis using certified sieves or particle size analysis using laser diffraction.

The analysis by means of certified sieves (i.e. perforated plates made of stainless steel) is carried out, for example, by means of a vibrating platform with n sieves assembled one over the other in a sieve holder container arranged above the vibrating platform (for example, frequency of about 3000 cycles/min). Each sieve in the sieve holder container has a different size (for example sieves from 250 μm to 2500 μm) and said sieves are positioned one over the other so that the larger sieves are arranged in the upper part of the container and the smaller sieves in the lower part of the container. The container is vibrated and a certain amount of a powder or granules is poured onto the upper sieve: the particles passing through the upper sieves reach the lower sieves or beyond. The operation ends when no evident separation occurs anymore. Stopping the powder on a sieve of a certain size determines its particle size. The sieves are quality certified: the certificate of conformity certifies that the mesh, materials used, dimensions and production process comply with the requirements.

The analysis of the size of solid particles—using the laser diffraction technique—is based on the principle that the particles illuminated by a laser beam diffuse the light at an angle related to the size thereof (the angle increases as the particle size decreases). The average diameter is determined based on the surface/volume ratio, using the parameter D (De Brouckere mean diameter—equation). The dimensional distribution is identified by the following parameters: D (0.1), D (0.5), D (0.9), which represent the cumulative distribution diameters of 10%, 50% and 90% of the total particles.

Furthermore, the gradual release of the amino acids in the intestinal tract over time by the composition of the invention in the form of granules, partly due to the embedding of the amino acid in said lipid matrices and partly due to the variation of the particle size, makes the amino acid bioavailable in the plasma at a constant percentage over a period of time comprised in the range from 2 hours to 24 hours. Said constant blood bioavailability over time allows to avoid fluctuations in bioavailability between meals.

In the context of the present invention, the term “bioavailability” is used to indicate the “relative bioavailability”, such as fraction of a compound under analysis (e.g. compound according to the invention) in the systemic circulation following the oral administration thereof in comparison with the fraction of a comparison compound (e.g. a feed or a composition not according to the invention) in the systemic circulation following the oral administration thereof. Said relative bioavailability of the compound under analysis can be expressed as a percentage considering 100% the fraction absorbed in the blood of the comparison compound: in this case, the percentage expressing the relative bioavailability of the compound under analysis may be less than 100% (lower bioavailability with respect to the comparison compound) or higher than 100% (higher bioavailability with respect to the comparison compound). Alternatively, said relative bioavailability of the compound under analysis can be expressed as a percentage difference with respect to the 1 (or 100) value of the blood-absorbed fraction of the comparison compound. For example, the following method may be used to determine the bioavailability of a composition according to the invention comprising lysine and a phytocompound derivative and a lipid matrix (e.g. rapeseed oil): two animal study groups are prepared, group 1 is administered with 1 kg of feed containing 40% of proteins, of which said proteins contain 10% of lysine (1 kg feed=40 g of lysine); group 2 is administered with an amount of composition of the invention containing 40 g of lysine. At a set time, the blood is collected from the animals of group 1 and group 2 and the mean lysine value present in the blood (in short, amount of lysine) is determined (for example by HPLC-MS) for each group. The amount of lysine determined for group 1 is set as a value 1 or a value of 100%, the amount of lysine determined for group 2 is expressed as a percentage or percentage difference with reference to said value 1 or 100%. Thus, if the amount of lysine in the blood of the Group 2 animals is, for example, 1.2 μg/ml and the amount of lysine in the blood of the Group 1 animals is 1.0 μg/ml, the bioavailability (relative bioavailability) of lysine of the composition of the invention is 120% or 20% more with respect to the bioavailability of lysine administered through the feed.

Advantageously, in order to be effective in the treatment methods described in the present invention, the compositions of the invention are administered to an animal in need in a daily dose comprising thymol in an amount (mg/kg of feed) comprised in the range from 5 mg/kg to 5000 mg/kg, preferably from 10 mg/kg to 2000 mg/kg, more preferably from 15 mg/Kg to 1000 mg/Kg.

The aforementioned daily doses may be administered to the subject in need in a single dose (one dose) or in repeated doses, for example two, three or four daily doses.

Lastly, forming an object of the present invention is the use of said feed or a feed additive comprising the composition or mixture M of the present invention, according to one of the described embodiments, for weaning or supporting the initial growth of a monogastric animal (mammalian or non-mammalian); preferably a monogastric mammal, more preferably pigs, or, alternatively, preferably a non-mammalian monogastric animal, such as poultry animals, fish or crustaceans.

In order to achieve the object of the present invention, the components (or active components) of the mixture M of the invention, such as thymol, components of group (I), (II) and/or (III), may be administered to an animal in need also separately and sequentially, and in any order; for example, in a close sequence over time (from about 0 minutes to 30 minutes) or in a non-close sequence over time (from 1 hour to about 4 or 6 or 8 or 12 hours), and administered at the same or different frequency. When said active components of the mixture M of the invention are administered in a single composition, said single composition corresponds to the composition of the present invention.

Unless specified otherwise, the expression composition or mixture or other comprising a component at an amount “comprised in a range from x to y” is used to indicate that said component can be present in the composition or other at all the amounts present in said range, even though not specified, extremes of the range comprised.

Unless specified otherwise, the indication that a composition “comprises” one or more components or substances means that other components or substances can be present besides the one, or the ones, indicated specifically.

In the context of the present invention, the expression “treatment method” is used to indicate an intervention on a subject in need, comprising the administration of the bacterial strain or of a composition of the invention with the aim of eliminating, reducing/decreasing or preventing a disease or ailment and the symptoms or disorders thereof.

The expression “therapeutically effective amount” refers to the amount of active compound or mixture of active components that elicits the biological or medicinal response in a tissue, system, animal, or human being that is sought and defined by a person skilled in the art (for example, a researcher, veterinarian, or physician).

In preferred examples of the composition of the invention according to said second embodiment (FR-II), said composition comprises: said (i) mixture of active components, said (ii) lipid matrix comprises or, alternatively, consists of: a triglyceride or a fatty acid or a wax and a mixture thereof (as defined in the context of the present invention) preferably rapeseed oil, palm oil and soybean oil, and, optionally, said (iii) at least one additive and/or excipient,

wherein said (i) mixture of active components is selected from a group comprising or, alternatively, consisting of:

FR-II-1: lysine and thymol, methionine and thymol, tryptophan and thymol, threonine and thymol, leucine and thymol, valine and thymol, isoleucine and thymol, arginine and thymol, histidine and thymol, phenylalanine and thymol;

FR-II-2: lysine and methionine and thymol, lysine and tryptophan and thymol, lysine and threonine and thymol, lysine and leucine and thymol, lysine and valine and thymol, lysine and isoleucine and thymol, lysine and arginine and thymol, lysine and histidine and thymol, lysine and phenylalanine and thymol;

FR-II-3: methionine and tryptophan and thymol, methionine and threonine and thymol, methionine and leucine and thymol, methionine and valine and thymol, methionine and isoleucine and thymol, methionine and arginine and thymol, methionine and histidine and thymol, methionine and phenylalanine and thymol;

FR-II-4: tryptophan and threonine and thymol, tryptophan and leucine and thymol, tryptophan and valine and thymol, tryptophan and isoleucine and thymol, tryptophan and arginine and thymol, tryptophan and histidine and thymol, tryptophan and phenylalanine and thymol;

FR-II-5: threonine and leucine and thymol, threonine and valine and thymol, threonine and isoleucine and thymol, threonine and arginine and thymol, threonine and histidine and thymol, threonine and phenylalanine and thymol;

FR-II-6: leucine and valine and thymol, leucine and isoleucine and thymol, leucine and valine and isoleucine and thymol, valine and isoleucine and thymol, leucine and arginine and thymol, leucine and histidine and thymol, leucine and phenylalanine and thymol;

FR-II-7: arginine and valine and thymol, arginine and isoleucine and thymol, arginine and histidine and thymol, arginine and phenylalanine and thymol;

FR-II-8: lysine and methionine and tryptophan and thymol, lysine and methionine and threonine and thymol, lysine and methionine and leucine and thymol, lysine and methionine and valine and thymol, lysine and methionine and isoleucine and thymol, lysine and methionine and arginine and thymol, lysine and methionine and histidine and thymol, lysine and methionine and phenylalanine and thymol; lysine and methionine and leucine and valine and isoleucine and thymol; lysine and methionine and valine and isoleucine and thymol;

FR-II-9: lysine and thymol and carvacrol, lysine and thymol and eugenol, lysine and thymol and capsaicin, lysine and thymol and tannins, lysine and thymol and verbascoside, lysine and thymol and saponins;

FR-II-10: methionine and thymol and carvacrol, methionine and thymol and eugenol, methionine and thymol and capsaicin, methionine and thymol and tannins, methionine and thymol and verbascoside, methionine and thymol and saponins;

FR-II-11: tryptophan and thymol and carvacrol, tryptophan and thymol and eugenol, tryptophan and thymol and capsaicin, tryptophan and thymol and tannins, tryptophan and thymol and verbascoside, tryptophan and thymol and saponins;

FR-II-12: threonine and thymol and carvacrol, threonine and thymol and eugenol, threonine and thymol and capsaicin, threonine and thymol and tannins, threonine and thymol and verbascoside, threonine and thymol and saponins;

FR-II-13: arginine and thymol and carvacrol, arginine and thymol and eugenol, arginine and thymol and capsaicin, arginine and thymol and tannins, arginine and thymol and verbascoside, arginine and thymol and saponins;

FR-II-14: leucine and thymol and carvacrol, leucine and thymol and eugenol, leucine and thymol and capsaicin, leucine and thymol and tannins, leucine and thymol and verbascoside, leucine and thymol and saponins;

FR-II-15: lysine and methionine and thymol and carvacrol, lysine and methionine and thymol and eugenol, lysine and methionine and thymol and capsaicin, lysine and methionine and thymol and tannins, lysine and methionine and thymol and verbascoside, lysine and methionine and thymol and saponins;

FR-II-16: lysine and methionine and tryptophan and thymol and carvacrol, lysine and methionine and tryptophan and thymol and eugenol, lysine and methionine and tryptophan and thymol and capsaicin, lysine and methionine and tryptophan and thymol and tannins, lysine and methionine and tryptophan and thymol and verbascoside, lysine and methionine and tryptophan and thymol and saponins;

FR-II-17: lysine and methionine and leucine and thymol and carvacrol, lysine and methionine and leucine and thymol and eugenol, lysine and methionine and leucine and thymol and capsaicin, lysine and methionine and leucine and thymol and tannins, lysine and methionine and leucine and thymol and verbascoside, lysine and methionine and leucine and thymol and saponins;

FR-II-18: lysine and methionine and threonine and thymol and carvacrol, lysine and methionine and threonine and thymol and eugenol, lysine and methionine and threonine and thymol and capsaicin, lysine and methionine and threonine and thymol and tannins, lysine and methionine and threonine and thymol and verbascoside, lysine and methionine and threonine and thymol and saponins;

FR-II-19: lysine and methionine and arginine and thymol and carvacrol, lysine and methionine and arginine and thymol and eugenol, lysine and methionine and arginine and thymol and capsaicin, lysine and methionine and arginine and thymol and tannins, lysine and methionine and arginine and thymol and verbascoside, lysine and methionine and arginine and thymol and saponins;

FR-II-20: lysine and tryptophan and thymol and carvacrol;

FR-II-21: methionine and tryptophan and thymol and carvacrol;

lysine and leucine and thymol and carvacrol; methionine and leucine and thymol and carvacrol; tryptophan and leucine and thymol and carvacrol;

FR-II-22: lysine and valine and isoleucine and thymol, lysine and valine and isoleucine and thymol and carvacrol; methionine and valine and isoleucine and thymol, methionine and valine and isoleucine and thymol and carvacrol; tryptophan and valine and isoleucine and thymol, tryptophan and valine and isoleucine and thymol and carvacrol; leucine and valine and isoleucine and thymol and carvacrol; lysine and methionine and valine and isoleucine and thymol and carvacrol; lysine and leucine and valine and isoleucine and thymol, lysine and leucine and valine and isoleucine and thymol and carvacrol; methionine and leucine and valine and isoleucine and thymol, methionine and leucine and valine and isoleucine and thymol and carvacrol; tryptophan and leucine and valine and isoleucine and thymol, tryptophan and leucine and valine and isoleucine and thymol and carvacrol; leucine and valine and isoleucine and thymol and carvacrol; lysine and methionine and leucine and valine and isoleucine and thymol, lysine and methionine and leucine and valine and isoleucine and thymol and carvacrol.

In preferred examples of the composition of the invention according to FR-II, said composition comprises: said (i) mixture of active components, said (ii) lipid matrix comprising or, alternatively, consisting of rapeseed oil and, optionally, said (iii) at least one additive and/or excipient (preferably coating additives), wherein said (i) mixture of active components is selected from a group comprising or, alternatively, consisting of what is listed in FR-II-1, FR-II-2, FR-II-3, FR-II-4, FR-II-5, FR-II-6, FR-II-7, FR-II-8, FR-II-9, FR-II-10, FR-II-11, FR-II-12, FR-II-13, FR-II-14, FR-II-15, FR-II-16, FR-II-17, FR-II-18, FR-II-19, FR-II-20, FR-II-21 and FR-II-22; preferably what is listed in FR-II-1.

In preferred examples of the composition of the invention according to FR-II, said composition comprises: said (i) mixture of active components, said (ii) lipid matrix comprises or, alternatively, consists of palm oil and, optionally, said (iii) at least one additive and/or excipient (preferably coating additives), wherein said (i) mixture of active components is selected from a group comprising or, alternatively, consisting of what is listed in FR-II-1, FR-II-2, FR-II-3, FR-II-4, FR-II-5, FR-II-6, FR-II-7, FR-II-8, FR-II-9, FR-II-10, FR-II-11, FR-II-12, FR-II-13, FR-II-14, FR-II-15, FR-II-16, FR-II-17, FR-II-18, FR-II-19, FR-II-20, FR-II-21 and FR-II-22; preferably what is listed in FR-II-1.

In preferred examples of the composition of the invention according to FR-II, said composition comprises: said (i) mixture of active components, said (ii) lipid matrix comprises or, alternatively, consists of soybean oil and, optionally, said (iii) at least one additive and/or excipient (preferably coating additives), wherein said (i) mixture of active components is selected from a group comprising or, alternatively, consisting of what is listed in FR-II-1, FR-II-2, FR-II-3, FR-II-4, FR-II-5, FR-II-6, FR-II-7, FR-II-8, FR-II-9, FR-II-10, FR-II-11, FR-II-12, FR-II-13, FR-II-14, FR-II-15, FR-II-16, FR-II-17, FR-II-18, FR-II-19, FR-II-20, FR-II-21 and FR-II-22; preferably what is listed in FR-II-1.

Preferably, in said embodiments of the invention comprising from FR-II-1 to FR-II-22, said (i) mixture comprises saponins when said non-mammalian monogastric animal is a poultry animal.

Preferably, in said embodiments of the invention comprising from FR-II-1 to FR-II-22, said (i) mixture comprises arginine or histidine when said non-mammalian monogastric animal is a fish or crustacean.

In said second embodiment (FR-II) of the composition of the present invention, preferably, in said (i) mixture of active components, the weight ratio [(i.1) at least one amino acid:(i.2) at least one phytocompound derivative] is comprised in the range from 1:10 to 10:1, preferably from 10:1 to 10:5, more preferably from 10:1 to 10:3.

Preferred aspects (FR-I-no) of said first embodiment of the invention (FR-I) are reported below: FR-I-1. A composition for use in a method for the preventive and/or curative treatment of inflammatory and/or functional intestinal disease or of a related symptom by modulating the receptors and/or enzymes of the endocannabinoid system, in a human being, or in a monogastric animal, or in birds or in fish, wherein said composition comprises: a mixture M comprising or, alternatively, consisting of thymol; and, optionally, at least one acceptable pharmaceutical or food grade additive and/or excipient.

FR-I-2. The composition for use according to FR-I-1, wherein said composition is for use in a monogastric mammal, such as in pigs; preferably a monogastric animal or monogastric mammal in the weaning phase, like in weaning pigs.

FR-I-3. The composition for use according to any one of FR-I 1 or 2, wherein said disease or symptom is selected from: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, irritable bowel syndrome (IBS), IBS with diarrhoea, IBS with constipation, IBS with alternating constipation and diarrhoea, unclassified IBS, dyspepsia, nausea, vomiting, constipation, diarrhoea, abdominal bloating, tympanites and physical fatigue.

FR-I-4. The composition for use according to any one of FR-I 1-3, wherein said composition further comprises a controlled release lipid matrix embedding or incorporating said mixture M comprising or, alternatively, consisting of thymol wherein said controlled release lipid matrix comprises or, alternatively, consists of at least one saturated or unsaturated, free or esterified fatty acid having a number of carbon atoms comprised in the range from C10-C30, and/or at least one triglyceride having saturated or unsaturated fatty acid chains, having a number of carbon atoms comprised in the range from C6-C30 and/or at least one wax having a number of carbon atoms comprised in the range from C16-C36;

wherein said lipid matrix allows a gastroprotection and/or a controlled intestinal release of the mixture M, preferably of the thymol contained therein.

FR-I-5. The composition for use according to any one of FR-I 1-4, wherein said controlled release lipid matrix comprises or, alternatively, consists of at least one hydrogenated fatty acid of plant origin having a number of carbon atoms comprised in the range from C14-C24 and/or at least one hydrogenated triglyceride of plant origin having a number of carbon atoms comprised in the range from C14-C24 and/or at least one wax of animal origin having a number of carbon atoms comprised in the range from C24-C36; preferably, wherein said fatty acid, triglyceride or wax is selected from: palm oil, sunflower oil, corn oil, rapeseed oil, peanut oil, soybean oil, olive oil, beeswax, and mixtures thereof.

FR-I-6. The composition for use according to any one of FR-I 1-5, wherein said mixture M further comprises at least one further first active ingredient, preferably deriving from a phytocompound (botanicals), selected from group (I) comprising or, alternatively, consisting of carvacrol, eugenol, capsaicin, turmeric, vanillin, cinnamaldehyde, diallyl disulfide, camphor, limonene, rosmarinic acid, p-cymene, γ-terpinene, α-pinene, α-thujone, 1,8-cineole, verbascoside, tannins and mixtures thereof.

FR-I-7. The composition for use according to any one of FR-I 1-6, wherein said composition comprises thymol and carvacrol, or thymol and vanillin, or thymol, carvacrol and capsaicin, or thymol, carvacrol and cinnamaldehyde, or thymol, eugenol and verbascoside.

FR-I-8. The composition for use according to any one of FR-I 1-7, wherein the mixture M contained in said composition comprises, besides thymol and, optionally, said further first active ingredient (botanicals) selected from group (I), an organic acid or a salt thereof with an alkaline or alkaline earth metal cation, wherein said organic acid is selected from group (II) comprising or, alternatively, consisting of lactic acid, malic acid, benzoic acid, fumaric acid, sorbic acid, citric acid, octanoic acid, heptanoic acid, butyric acid, dodecanoic acid and mixture thereof; preferably it may comprise thymol and sorbic acid, or thymol, sorbic acid and citric acid, or thymol and benzoic acid, or thymol, sorbic acid, citric acid and benzoic acid, or thymol, carvacrol and sorbic acid, or thymol, carvacrol, sorbic acid and citric acid, or thymol, carvacrol, cinnamaldehyde and sorbic acid, or thymol, carvacrol, cinnamaldehyde, sorbic acid and citric acid, or thymol and butyric acid, or thymol, citric acid and dodecanoic acid.

FR-I-9. The composition for use according to any one of FR-I 1-8, wherein said mixture M further comprises at least one further second active ingredient selected from group (Ill) consisting of: bacterial strains or probiotic bacterial strains belonging to the genus Lactobacillus, Bidobacterium, Streptococcus, Leuconostoc, Pediococcus, Enterococcus, Saccharomyces; and/or prebiotics, such as for example inulin, lactulose, lactitol, mannan-oligosaccharides, fructigosaccharides and galacto-oligosaccharides, tributyrin; and/or salts of metals, such as zinc and copper; and mixtures thereof.

FR-I-10. The composition for use according to any one of FR-I 1-9, wherein said composition is a feed or a feed additive for monogastric animals and/or monogastric animals in weaning or initial growth phase; preferably for monogastric mammals, more preferably for pigs.

### Experimental Part I

The purpose of this study was to study the presence of markers of the endocannabinoid system and of the pig gut chemosensing system and, secondly, to determine how thymol modulates these markers.

### Method

### Ethics Statement

The study was conducted at the facilities of the Research Centre for Animal Production and Environment (CER-O), which is Good Laboratory Practices-certified and operates according to the Procedure of Animal Protection and Welfare (directive No 86/609/EEC) Animals used in the study were raised and treated according to European Union directive 2010/63/EU. The study was authorized by the Italian Ministry of Health according to art. 31 of the Italian legislative decree no 26/2014 and on Commission recommendation 2007/526/EC, which covers the housing and care of animals used for experimental and other scientific purposes (authorization no 341/2017-PR issued May 3, 2017). Animals were obtained from a breeding farm in Cascina Mandellina, Bergamo, Italy.

### Animals and Diets

One hundred and fifty piglets (Duroc—Large White) weaned at 28 days of age and with a body weight (BW) of 7.71±1.00 kg were divided into 40 pens (4 piglets per pen, castrated males and females were placed in separate pens) and randomly assigned to one of the following experimental groups (n=32): control group fed with the basal diet (T1), a group fed with the basal diet supplemented with 25.5 mg of thymol/kg feed (T2), a group fed with the basal diet supplemented with 51 mg of thymol/kg feed (T3), a group fed with the basal diet supplemented with 153 mg of thymol/kg feed (T4) and a group fed with the basal diet supplemented with 510 mg of thymol/kg feed (T5). Thymol was provided in a form embedded (microencapsulated) in a lipid matrix (Vetagro SpA, Reggio Emilia, Italy). Concentrations of thymol were selected to meet or exceed the upper limit of inclusion in food and feed established by the European Agency for the Evaluation of Medicinal Products and for the feed. The basal feed was formulated to meet or exceed the nutritional requirements of pigs according to the National Research Council, and feed and water were provided ad libitum (the composition of the basal diet is reported in Table 1). The health condition of the animals was monitored during the study. The piglets were individually weighed at the beginning (day 0) and end (day 14) of the study. Growth parameters, such as FI, ADFI, ADG and F:G (FI—feed intake; ADFI—Average daily feed intake; ADG—Average daily gain; F:G—Feed to gain ratio), were measured in the animals housed in each pen on day 14 (d14) of the experiment.

TABLE 1
Ingredient (% dry matter)

| | |
|---|---|
| Corn meal | 59.25 |
| Soybean meal, 44% | 21.90 |
| Sweet milk whey | 8.00 |
| Fishmeal (Herring 999) | 7.00 |
| Soybean oil | 1.98 |
| Calcium carbonate | 0.35 |
| Vitamin and mineral premix | 0.25 |
| L-lysine HCl | 0.54 |
| NaCl | 0.16 |
| L-threonine | 0.24 |
| DL-methionine | 0.26 |
| L-tryptophan | 0.08 |
| Total | 100 |

At the end of the study, 8 animals from each treatment group were sacrificed, samples were collected, and analysed. Duodenal and ileal mucosal scrapings were collected. The duodenum and ileum were longitudinally cut to expose the mucosa, washed with a phosphate-buffered saline solution to remove mucus and digesta, then scraped gently, packed, immediately frozen in liquid nitrogen and stored at −80° C. until the analyses of gene and protein expression.

### Gene Expression Analysis

Gene expression was analysed using the method reported by Herfel et al. [The Journal of Nutrition. 2011; 141:2139-45]. Duodenal and ileal scraping samples obtained on d14 of the study were disrupted by grinding them in liquid nitrogen with mortar and pestle, and then homogenised using a TissueLyser (Qiagen, Hilden, Germany). Total RNA was isolated using a NucleoSpin® RNA Kit (Macherey-Nagel, Duren, Germany) according to the manufacturer's instructions. Genomic DNA contamination was removed by treating the samples with the deoxyribonuclease supplied in the extraction kit (rDNase, RNase-free; Macherey-Nagel). The RNA yield and quality were determined spectrophotometrically by measuring the absorbance at 260 and 280 nm (A260 and A280 nm, respectively) (Microvolume Mode with SmartPath® Technology, Denovix). One microgram of RNA was reverse transcribed using the iScript cDNA Synthesis Kit (Bio-Rad Laboratories Inc., Hercules, Calif., USA) according to the manufacturer's instructions. Real-time PCR was performed using an iCycler Thermal Cycler system and SybrGreen Supermix (Bio-Rad Laboratories Inc.). The thermocycling protocol provided for an initial denaturation step for 1 minute and 30 seconds at 95° C., followed by 40 denaturation cycles at 95° C. for 15 seconds, and 30 seconds of annealing and extension at 60° C. After amplification, a melt curve analysis was performed for all samples, with slow heating from 55° C. to 95° C. at a rate of 0.5° C./s to validate the absence of non-specific products. Gene expression was normalised to a housekeeping gene (HK) encoding portions of porcine ribosomal subunit 60 S, in particular ribosomal protein L35 (RPL35). The average threshold cycle (CT) was determined for each gene of interest, and the geometric average was calculated for HK by assuming that CT is the number of cycles required to reach a fixed arbitrary threshold. Delta CT was calculated, then a modification of the 2^{-ΔΔCT} method [Livak K J, et al., Methods 2001; 25:402-8] was used to analyse the relative expression (fold changes), which was calculated relative to the control group. The sequences, accession numbers in the EMBL database/GenBank, expected product lengths and references for porcine primers are reported in Table 2. Primer oligonucleotides for CB1, DGL-β and OR1G1 were designed using the Primer-BLAST tool (NCBI National Center for Biotechnology Information, www.ncbi.nlm.nih.gov). Primers were obtained from Life Technologies (Life Technologies Italia).

TABLE 2

| | Primer sequence (F and R) | | |
|---|---|---|---|
| Gene | 5′ → 3′ | bp | Accession No |
| CB1 | F: TTCCCCACTTCTTTTCCGCC | 208 | XM_013992672.2 |
| | R: GGGAGTCCCTTCGCATCC | | |
| CB2 | F: TTTATAGCCTGGCCTCCCCT | 240 | XM_021095530.1 |
| | R: TTTTCCCGTCTGCCTCTGTC | | |
| FAAH | F: TGCCACCGTGCAAGAAAATG | 234 | XM_013999418.2 |
| | R: CCACTGCCCTAACAACGACT | | |
| DGL-α | F: GAAACCAAACACGCCTCCAC | 211 | XM_021082924.1 |
| | R: CAACCCAGCAGCAAAGGAAC | | |
| DGL-β | F: TTTGTAATCCCGGACCACGG | 255 | XM_021086077.1 |
| | R: GACCTGCCGAGGAATACGGA | | |
| TRPV1 | F: TCACCAACAAGAAGGGGCTC | 116 | XM_005669121.1 |
| | R: GGATAGGTGCCTGCACTCAG | | |
| OR1G1 | F: CTTGGTTTGTGTGCTCTGCC | 96 | XM_013990010.1 |
| | R: GAAAAGGCTTTCCGCTTCCC | | |

bp: product length; F: forward; R: Reverse

### Statistical Analysis

Animals were blocked in a completely randomised design and data were analysed using GraphPad Prism® software (GraphPad Software, Inc., La Jolla, Calif., USA). Data were analysed using one-way ANOVA followed by Tukey's post hoc test to detect differences among treatments. The pen was the experimental unit for growth performance, whereas the pig was the experimental unit for gene expression. Differences were considered significant at P<0.05, and trends were defined at 0.05≤P<0.1.

### Results

### Growth Performance

The piglets maintained a good health conditions during the experiment and no mortality was recorded.

During the experiment, differences in body weight (BW), feed intake (FI), average daily feed intake (ADFI), average daily gain (ADG), and feed to gain ratio (F:G) were not observed among the treatment groups (T2, T3, T4 e T5; data not shown).

### Endocannabinoid System (ECS)

FIG. 1 summarises gene expression data for cannabinoid receptors in the duodenal and ileal mucosa at day 14 (d14). Cannabinoid receptor 1 and 2 mRNAs were detected in both the duodenal and ileal mucosa. The level of the CB1 mRNA was significantly increased in the duodenum of group T5 (P=0.0209) and in the ileum of groups T4 and T5 (P=0.0054) compared to the control group. Significantly increased levels of the CB2 mRNA were detected in both the duodenum and ileum of groups T4 and T5 compared to the control group (P=0.004 and P=0.0162 respectively). Data on gene expression for ECS enzymes are reported in FIG. 2. The presence of mRNA for all the enzymes tested was confirmed. Differences in FAAH mRNA levels were not observed in duodenum, while FAAH mRNA levels were significantly increased in the ileum of group T4 compared to the control group (P=0.0028).

### Gut Chemosensing System

The results for the gut chemosensing are reported in FIG. 3. As regards the gut chemosensing markers, both TRPV1 and OR1G1 (Olfactory receptor 1G1) mRNAs were detected in the duodenal and ileal mucosa. Furthermore, completion of 510 mg thymol/kg of feed (T5) increased the TRPV1 mRNA level in the duodenum (P=0.0382), while increased TRPV1 mRNA levels were observed in the ileum of groups T4 and T5 compared to the control group (P=0.0183). The OR1G1 mRNA was expressed at higher levels in the duodenum of animals fed with feed supplemented with 510 mg thymol/kg feed (T5) (P=0.0210) and in the ileum of animals fed with 153 mg thymol/kg feed (T4) (P=0.0235) compared to the control group.

### Conclusions

In conclusion, the data of the present study not only confirm the presence of markers of the endocannabinoid system (ECS) and of the gut chemosensing n the duodenal and ileal mucosa of the piglets, but it also demonstrates that thymol modulates the gene expression of these markers. Thymol increases the expression of the mRNAs encoding the CB1 and CB2 receptors both in the duodenum and ileum. Thymol also modulates mRNA levels of enzymes involved in biosynthesis and degradation of endocannabinoid molecules (e.g. FAAH). Furthermore, the upregulation of OR1G1 and TRPV1 (chemosensory receptors) performed by thymol in the intestine demonstrates a possible role of thymol as a feeding additive in the enhancement of the intestinal health of the animal, particularly in the weaning and growth phase.

### Experimental Part II

A method for measuring the plasma bioavailability of amino acids in a monogastric animal following the administration of a composition according to the present invention (comprising at least one amino acid, at least one phytocompound and a lipid matrix) consists of:
administer the following diets to 3 experimental groups of an animal species under study (for example, chicken or fish):

group 1. a control diet (for example soy-based),

group 2. a diet added with a comparison composition: composition comprising amino acids and phytocompound derivatives in the absence of a lipid matrix (non-embedded active components), and

group 3. a diet added with a composition according to the invention: composition comprising amino acids and phytocompound derivatives in the presence of a lipid matrix (embedded active components)
collect blood samples from the animals under study and obtain the plasma fraction. The samples are collected at different time-points after the administration of the diets (from 10 minutes up to 360 minutes after the administration) and the presence of one or more amino acids in the obtained plasma fractions is evaluated by means of the LC/MS-MS (Liquid Chromatography with tandem Mass Spectrometry) plasma amino acid assay method.

### Experimental Part III

Table 3 shows the values of the experimental study which analysed the release of phytocompound derivatives embedded in a lipid matrix in the form of granules (composition according to the invention). As the data show, the release is a function of the time and size of the granules, the larger the granule size, the slower the release of the active ingredient.

The data were obtained by incubating 1 gram of granules of different sizes in a buffer simulating the intestinal pH conditions. At each time point (1 h, 2 h, 4 h) the phytocompound derivatives still present in the granules were quantified (by means of HPLC), and the release percentages were calculated by difference. The experiment was triplicated.

TABLE 3

| Particle size | Fraction at 1 hour | Fraction at 2 hours | Fraction at 3 hours |
|---|---|---|---|
| 2000-2500 um | 0 | 0 | 0 |
| 1500-2000 um | 0 | 17% | 8% |
| 1000-1500 um | 0 | 18% | 3% |
| 500-1000 um | 0 | 15% | 20% |
| 50-500 um | 26% | 36% | 48% |

### Examples

Representative examples of compositions of the invention according to the second embodiment (FR-II) are shown in Table 4

TABLE 4

| | AA1 | AA2 | AA3 | der-FT1 | der-FT2 | Add | Matrix oil |
|---|---|---|---|---|---|---|---|
| | (%) | (%) | (%) | (%) | (%) | (%) | (%) |
| Comp 1 | Lys | Met | — | a (5%) | b (2%) | (5%) | rapeseed |
| | (18%) | (15%) | | | | | (55%) |
| Comp 2 | Lys | Met | Thr | a (10%) | c (5%) | (3%) | rapeseed |
| | (10%) | (5%) | (5%) | | | | (62%) |
| Comp 3 | Lys | Val | IsoLeu | b (12%) | c (2%) | (1%) | palm |
| | (15%) | (10%) | (10%) | | | | (50%) |
| Comp 4 | Thr | Met | Trp | b (4%) | d (10%) | (3%) | palm |
| | (10%) | (5%) | (8%) | | | | (60%) |
| Comp 5 | Thr | Val | IsoLeu | d (10%) | g (6%) | (10%) | soy |
| | (10%) | (5%) | (5%) | | | | (54%) |
| Comp 6 | Arg | Met | — | c (15%) | f (2%) | (3%) | soy |
| | (10%) | (15%) | | | | | (55%) |
| Comp 7 | Lys | Met | His | a (8%) | e (2%) | (15%) | rapeseed |
| | (10%) | (15%) | (5%) | | | | (45%) |

(%): weight/weight composition.
AA: Amino acid.
der-FT: phytocompound derivative [(a) thymol, (b) carvacrol, (c) eugenol, (d) capsaicin, (e) tannins, (f) verbascosde, (g) saponins].
Add: additive.

Preferred embodiments FRn of the present invention are reported below.

FR1. A composition for use in a method for preventive and/or curative treatment of an inflammatory and/or functional intestinal disease or symptom in a mammalian or non-mammalian monogastric subject, wherein said mammalian monogastric subject is a human subject or a pig and wherein said non-mammalian monogastric subject is a poultry animal or a fish or a crustacean, wherein said composition comprises:

(i) a mixture of active components comprising, or alternatively, consisting of:
thymol and
at least one amino acid or an acceptable pharmaceutical or food grade salt thereof, wherein said amino acid is selected from a group comprising or, alternatively, consisting of: lysine, methionine, tryptophan, threonine, leucine, valine, isoleucine, arginine, phenylalanine and a mixture thereof;

and, optionally, said composition further comprises (iii) at least one acceptable pharmaceutical or food grade additive and/or excipient;

wherein said thymol modulates the receptors and/or enzymes of the endocannabinoid system.

FR2. The composition for use according to FR1, wherein said composition further comprises (ii) a lipid matrix embedding said (i) mixture of active components,

wherein said lipid matrix comprises or, alternatively, consists of:

at least one saturated or unsaturated, free or esterified fatty acid having a number of carbon atoms comprised in the range C10-C30, and/or

at least one triglyceride having saturated or unsaturated fatty acid chains, having a number of carbons comprised in the range C6-C30 and/or

at least one wax having a number of carbon atoms comprised in the range C16-C36;

wherein said composition is a solid composition in the form of granules having the following particle size distribution percentage with respect to 100 granules:
when said subject is a human subject or a pig: from 5% to 10% of granules having an average particle size from 50 μm to 500 μm, from 25% to 35% of granules having a particle size from 500 μm to 1000 μm, from 45% to 55% of granules having a particle size from 1000 μm to 1500 μm, from 20% to 30% of granules having a particle size from 1500 μm to 2000 μm, from 0.1% to 1% of granules having a particle size from 2000 μm to 2500 μm;
when said animal is a fish or a crustacean: from 10% to 20% of granules having a particle size from 50 μm to 250 μm, from 45% to 55% of granules having a particle size from 250 μm to 400 μm, from 20% to 30% of granules having a particle size from 400 μm to 500 μm, from 5% to 15% of granules having a particle size from 500 μm to 2500 μm;
when said animal is a poultry animal: from 1% to 10% of granules having an average particle size from 50 μm to 500 μm, from 45% to 55% of granules having a particle size from 500 μm to 1000 μm, from 35% to 45% of granules having a particle size from 1000 μm to 1500 μm, from 1% to 9% of granules having a particle size from 1500 μm to 2000 μm, from 0.1% to 1% of granules having a particle size from 2000 μm to 2500 μm;

wherein said composition is administered to said subject through oral route,

wherein said (ii) lipid matrix is capable of providing a gastroprotection of said thymol and said at least one amino acid,

wherein said (ii) lipid matrix is capable of providing a controlled release of said thymol and said at least one amino acid within a time range comprised from 30 minutes to 8 hours in the intestinal tract.

FR3. The composition for use according to FR1 or FR2, wherein said (ii) lipid matrix, comprising said at least one fatty acid and/or said at least one triglyceride, is selected from a group comprising or, alternatively, consisting of: rapeseed oil, palm oil, soybean oil and a mixture thereof; preferably rapeseed oil or a mixture thereof when said subject is a poultry animal, a fish or a crustacean; preferably soybean oil or a mixture thereof when said subject is a human subject or a pig.

FR4. The composition for use according to any one of FR1-3,

wherein said (i) mixture of active components comprises or, alternatively, consists of: lysine and thymol, and

wherein said (ii) lipid matrix comprises or, alternatively, consists of: rapeseed oil or palm oil or soybean oil or a mixture thereof.

FR5. The composition for use according to any one of FR1-3,

wherein said (i) mixture of active components comprises or, alternatively, consists of: methionine and thymol, and

wherein said (ii) lipid matrix comprises or, alternatively, consists of: rapeseed oil or palm oil or soybean oil or a mixture thereof.

FR6. The composition for use according to any one of FR1-3,

wherein said (i) mixture of active components comprises or, alternatively, consists of: tryptophan and thymol, and

wherein said (ii) lipid matrix comprises or, alternatively, consists of: rapeseed oil or palm oil or soybean oil or a mixture thereof.

FR7. The composition for use according to any one of FR1-3,

wherein said (i) mixture of active components comprises or, alternatively, consists of: threonine and thymol, and

wherein said (ii) lipid matrix comprises or, alternatively, consists of: rapeseed oil or palm oil or soybean oil or a mixture thereof.

FR8. The composition for use according to any one of FR1-3,

wherein said (i) mixture of active components comprises or, alternatively, consists of: arginine and thymol, and

wherein said (ii) lipid matrix comprises or, alternatively, consists of: rapeseed oil or palm oil or soybean oil or a mixture thereof.

FR9. The composition for use according to any one of FR1-8, wherein said disease or symptom is selected from the group comprising or, alternatively, consisting of: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, celiac disease, irritable bowel syndrome (IBS), IBS with diarrhoea, IBS with constipation, IBS with alternating constipation and diarrhoea, unclassified IBS, dyspepsia, nausea, vomiting, constipation, diarrhoea, abdominal bloating, tympanites and physical fatigue.

FR10. Non-therapeutic use of a composition according to any one of FR1-9, for preparing a feed or a feed additive, wherein said feed is a feed for a mammalian or non-mammalian monogastric subject, wherein said mammalian monogastric subject is a pig and/or wherein said non-mammalian monogastric subject is a poultry animal, a fish or a crustacean.

1. A composition for use in a method for preventive and/or curative treatment of an inflammatory intestinal disease or symptom in a mammalian or non-mammalian monogastric subject,
wherein said mammalian monogastric subject is a human subject or a pig and wherein said non-mammalian monogastric subject is a poultry animal or a fish or a crustacean,
wherein said composition comprises:
(i) a mixture of active components comprising, or alternatively, consisting of:
thymol, and
at least one amino acid or an acceptable pharmaceutical or food grade salt thereof, wherein said amino acid is selected from a group consisting of: lysine, methionine, tryptophan, threonine, leucine, valine, isoleucine, arginine, phenylalanine and a mixture thereof;
and, optionally, said composition further comprises (iii) at least one acceptable pharmaceutical or food grade additive and/or excipient;
wherein said thymol modulates the receptors and/or enzymes of the endocannabinoid system,
wherein said composition further comprises (ii) a lipid matrix embedding said (i) mixture of active components,
wherein said lipid matrix comprises or, alternatively, consists of:
at least one saturated or unsaturated, free or esterified fatty acid having a number of carbon atoms comprised in the range C10-C30, and/or
at least one triglyceride having saturated or unsaturated fatty acid chains, having a number of carbons comprised in the range C6-C30 and/or
at least one wax having a number of carbon atoms comprised in the range C16-C36;
wherein said composition is a solid composition in the form of granules having the following particle size distribution percentage with respect to 100 granules:
when said subject is a human subject or a pig: from 5% to 10% of granules having a particle size from 50 μm to 500 μm, from 25% to 35% of granules having a particle size from 500 μm to 1000 μm, from 45% to 55% of granules having a particle size from 1000 μm to 1500 μm, from 20% to 30% of granules having a particle size from 1500 μm to 2000 μm, from 0.1% to 1% of granules having a particle size from 2000 μm to 2500 μm; or
when said animal is a fish or a crustacean: from 10% to 20% of granules having a particle size from 50 μm to 250 μm, from 45% to 55% of granules having a particle size from 250 μm to 400 μm, from 20% to 30% of granules having a particle size from 400 μm to 500 μm, from 5% to 15% of granules having a particle size from 500 μm to 2500 μm; or
when said animal is a poultry animal: from 1% to 10% of granules having an average particle size from 50 μm to 500 μm, from 45% to 55% of granules having a particle size from 500 μm to 1000 μm, from 35% to 45% of granules having a particle size from 1000 μm to 1500 μm, from 1% to 9% of granules having a particle size from 1500 μm to 2000 μm, from 0.1% to 1% of granules having a particle size from 2000 μm to 2500 μm;
wherein said composition is administered to said subject through oral route,
wherein said (ii) lipid matrix is capable of providing a gastroprotection of said thymol and said at least one amino acid,
wherein said (ii) lipid matrix is capable of providing a controlled release of said thymol and said at least one amino acid within a time range comprised from 30 minutes to 8 hours in the intestinal tract.

2. The composition for use according to claim 1, wherein said (ii) lipid matrix, comprising said at least one fatty acid and/or said at least one triglyceride, is selected from a group consisting of: rapeseed oil, palm oil, soybean oil and a mixture thereof; preferably rapeseed oil or a mixture thereof when said subject is a poultry animal, a fish or a crustacean; preferably soybean oil or a mixture thereof when said subject is a human subject or a pig.

3. The composition for use according to claim 1,
wherein said (i) mixture of active components comprises or, alternatively, consists of: lysine and thymol, and
wherein said (ii) lipid matrix comprises or, alternatively, consists of: rapeseed oil or palm oil or soybean oil or a mixture thereof.

4. The composition for use according to claim 1,
wherein said (i) mixture of active components comprises or, alternatively, consists of: methionine and thymol, and
wherein said (ii) lipid matrix comprises or, alternatively, consists of: rapeseed oil or palm oil or soybean oil or a mixture thereof.

5. The composition for use according to claim 1,
wherein said (i) mixture of active components comprises or, alternatively, consists of: tryptophan and thymol, and
wherein said (ii) lipid matrix comprises or, alternatively, consists of: rapeseed oil or palm oil or soybean oil or a mixture thereof.

6. The composition for use according to claim 1,
wherein said (i) mixture of active components comprises or, alternatively, consists of: threonine and thymol, and
wherein said (ii) lipid matrix comprises or, alternatively, consists of: rapeseed oil or palm oil or soybean oil or a mixture thereof.

7. The composition for use according to claim 1,
wherein said (i) mixture of active components comprises or, alternatively, consists of: arginine and thymol, and
wherein said (ii) lipid matrix comprises or, alternatively, consists of: rapeseed oil or palm oil or soybean oil or a mixture thereof.

8. The composition for use according to claim 1, wherein said disease or symptom is selected from the group comprising or, alternatively, consisting of: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, celiac disease, irritable bowel syndrome (IBS), IBS with diarrhoea, IBS with constipation, IBS with alternating constipation and diarrhoea, unclassified IBS, dyspepsia, nausea, vomiting, constipation, diarrhoea, abdominal bloating and, tympanites.

9. Non-therapeutic use of a composition according to claim 1 for preparing a feed or a feed additive, wherein said feed is a feed for a mammalian or non-mammalian monogastric subject, wherein said mammalian monogastric subject is a pig and/or wherein said non-mammalian monogastric subject is a poultry animal, a fish or a crustacean.

10. A method of treating a subject for an inflammatory disease, comprising administering the composition of claim 1 to the subject.

11. The method of claim 10, wherein the subject has a inflammatory disease of the gastrointestinal tract.

12. The method of claim 10, wherein the disease is selected from the group consisting of chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, celiac disease, irritable bowel syndrome (IBS), IBS with diarrhoea, IBS with constipation, IBS with alternating constipation and diarrhoea, unclassified IBS, dyspepsia, nausea, vomiting, constipation, diarrhoea, abdominal bloating and, tympanites.

13. The method of claim 10, wherein said (ii) lipid matrix, comprising said at least one fatty acid and/or said at least one triglyceride, is selected from a group consisting of: rapeseed oil, palm oil, soybean oil and a mixture thereof; preferably rapeseed oil or a mixture thereof when said subject is a poultry animal, a fish or a crustacean; preferably soybean oil or a mixture thereof when said subject is a human subject or a pig.

14. The method of claim 10, wherein said (i) mixture of active components comprises or, alternatively, consists of: lysine and thymol, and
wherein said (ii) lipid matrix comprises or, alternatively, consists of: rapeseed oil or palm oil or soybean oil or a mixture thereof.

15. The method of claim 10, wherein said (i) mixture of active components comprises or, alternatively, consists of: methionine and thymol, and
wherein said (ii) lipid matrix comprises or, alternatively, consists of: rapeseed oil or palm oil or soybean oil or a mixture thereof.

16. The method of claim 10, wherein said (i) mixture of active components comprises or, alternatively, consists of: tryptophan and thymol, and
wherein said (ii) lipid matrix comprises or, alternatively, consists of: rapeseed oil or palm oil or soybean oil or a mixture thereof.

17. The method of claim 10, wherein said (i) mixture of active components comprises or, alternatively, consists of: threonine and thymol, and
wherein said (ii) lipid matrix comprises or, alternatively, consists of: rapeseed oil or palm oil or soybean oil or a mixture thereof.

18. The method of claim 10, wherein said (i) mixture of active components comprises or, alternatively, consists of: arginine and thymol, and
wherein said (ii) lipid matrix comprises or, alternatively, consists of: rapeseed oil or palm oil or soybean oil or a mixture thereof.


SEQUENCE LISTING (14 sequences)

SEQ ID NO: 1 - DNA, 20 bp, Artificial Sequence
  Primer F CB1 (Accession N XM_013992672.2)
ttccccacttcttttccgcc

SEQ ID NO: 2 - DNA, 18 bp, Artificial Sequence
  Primer R CB1 (Accession N XM_013992672.2)
gggagtcccttcgcatcc

SEQ ID NO: 3 - DNA, 20 bp, Artificial Sequence
  Primer F CB2 (Accession N XM_021095530.1)
tttatagcctggcctcccct

SEQ ID NO: 4 - DNA, 20 bp, Artificial Sequence
  Primer R CB2 (Accession N XM_021095530.1)
ttttcccgtctgcctctgtc

SEQ ID NO: 5 - DNA, 20 bp, Artificial Sequence
  Primer F FAAH (Accession N XM_013999418.2)
tgccaccgtgcaagaaaatg

SEQ ID NO: 6 - DNA, 20 bp, Artificial Sequence
  Primer R FAAH (Accession N XM_013999418.2)
ccactgccctaacaacgact

SEQ ID NO: 7 - DNA, 20 bp, Artificial Sequence
  Primer F DGL- (Accession N XM_021082924.1)
gaaaccaaacacgcctccac

SEQ ID NO: 8 - DNA, 20 bp, Artificial Sequence
  Primer R DGL- (Accession N XM_021082924.1)
caacccagcagcaaaggaac

SEQ ID NO: 9 - DNA, 20 bp, Artificial Sequence
  Primer F DGL- (Accession N XM_021086077.1)
tttgtaatcccggaccacgg

SEQ ID NO: 10 - DNA, 20 bp, Artificial Sequence
  Primer R DGL- (Accession N XM_021086077.1)
gacctgccgaggaatacgga

SEQ ID NO: 11 - DNA, 20 bp, Artificial Sequence
  Primer F TRPV1 (Accession N XM_005669121.1)
tcaccaacaagaaggggctc

SEQ ID NO: 12 - DNA, 20 bp, Artificial Sequence
  Primer R TRPV1 (Accession N XM_005669121.1)
ggataggtgcctgcactcag

SEQ ID NO: 13 - DNA, 20 bp, Artificial Sequence
  Primer F OR1G1 (Accession N XM_013990010.1)
cttggtttgtgtgctctgcc

SEQ ID NO: 14 - DNA, 20 bp, Artificial Sequence
  Primer R OR1G1 (Accession N XM_013990010.1)
gaaaaggctttccgcttccc